(19) United States
(12) Patent Application Publication
(10) Pub. No.: US 2004/0258629 A1
(43) Pub. Date: Dec. 23, 2004

(54) Antiplaque confectionery dental composition

(76) Inventors: Thomas J. Boyd, Somerset, NJ (US); Guofeng Xu, Princeton, NJ (US); Abdul Gaffar, Princeton, NJ (US); David B. Viscio, Monmouth Jct, NJ (US)

Correspondence Address: Colgate-Palmolive Company, 909 River Road, P.O. Box 1343, Piscataway, NY, 08855-1343, US

(21) Appl. No.: 10/601,477

(22) Filed: Jun. 23, 2003

(51) Int. Cl. A61K 9/68; A61K 7/16

(52) U.S. Cl. 424/48; 424/49

(57) ABSTRACT

A confectionery composition delivering antiplaque agents to the oral cavity when applied thereto, the composition being comprised of a homogeneous mixture of a solid base material and an antibacterial ester having the formula

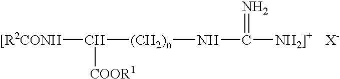

wherein R is an alkyl chain of 1 to 8 carbon atoms and R¹ is an alkyl chain of 6 to 30 carbon atoms and X is an anion.

### BACKGROUND OF THE INVENTION

1. Field of the Invention

The present invention relates to a confectionery composition which inhibits the formation of dental plaque on tooth surfaces and more particularly the invention relates to a confectionery composition which contains a small but effective amount of a an antibacterial ester compound which inhibits plaque formation and adhesion to tooth surfaces.

2. The Prior Art

Oral compositions such as toothpastes, gels and mouth washes are designed to loosen and remove plaque in conjunction with a regular toothbrushing regimen. Dental plaque is present to some degree, in the form of a film, on virtually all dental surfaces. It is a byproduct of microbial growth, and comprises a dense microbial layer consisting of a mass of microorganisms embedded in a polysaccharide matrix. Plaque itself adheres firmly to dental surfaces and is removed only with difficulty even through a rigorous brushing regimen. Moreover, plaque rapidly reforms on the tooth surface after it is removed. Plaque may form on any part of the tooth surface, and is found particularly at the gingival margin, in cracks in the enamel, and on the surface of dental calculus. The problem associated with the formation of plaque on the teeth lies in the tendency of plaque to build up and eventually produce gingivitis, periodontitis and other types of periodontal disease, as well as dental caries and dental calculus.

Plaque formation is an ongoing process. Although various oral care products are available to control plaque formation such as toothpastes and mouth rinse, the disadvantage of these products is that only a relatively short time during which the teeth are being brushed or the mouth is being rinsed is available for these preparations to take effect. A further disadvantage of these toothpaste and mouth rinse products is the general infrequency of use, that is, most dental hygiene products are used once or perhaps twice daily and seldom when they are most needed, e.g., after meals and snacks. Thus food deposits which build up as a result of eating throughout the day are left in the oral cavity for long periods of time thereby promoting microbial growth and formation of plaque on tooth surfaces.

A wide variety of antibacterial agents have been suggested in the art to retard plaque formation and the oral infections associated with plaque formation. For example, halogenated hydroxydiprrehyl ether compounds such as Triclosan are well known to the art for their antibacterial activity and have been used in oral compositions to counter plaque formation by bacterial accumulation in the oral cavity. Br. 1,352,420 discloses that the mono-N-higher aliphatic acyl arginine derivatives adhere to the mucosa in the oral cavity and possess an antibacterial activity against oral bacterial such as Lactobacillus, a main pathogen of dental caries and bacterium belonging to the genus staphylococcus, a main pathogen of alveolar pyorrhea.

U.S. Pat. No. 5,874,068 discloses an antiplaque effective mouthrinse containing a N^{α}-acyl acidic amino acid ester salt stabilized by the presence in the mouthrinse of monohydric alcohol such as ethanol, as aqueous compositions containing salts of N^{α}-alkyl-L-arginine alkyl esters undergo hydrolysis in aqueous environments.

A critical requirement, however, for these compositions is that they are stable and have a long shelf-life, which requirement has limited the use of these compositions because normally, the active agents incorporated in these compositions that provide oral care benefits such as plaque reduction are not stable under ambient conditions of humidity and temperature and as a result the agents quickly become degraded to concentrations of limited efficacy.

In view of the inconvenience of using toothpaste and mouth rinse products when away from home, the art is seeking portable products in the form of solid confections such as lozenges and chewing gums which can be used throughout the day, particularly after eating, and which provide antiplaque benefits comparable to those obtained by regular brushing with a toothpaste or use of a mouthrinse.

### SUMMARY OF THE INVENTION

In accordance with the present invention, there is provided a confectionery composition such as a lozenge or chewing gum comprised of a small but effective amount of a plaque reducing antibacterial ester having the formula.

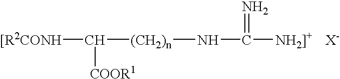

wherein R is an alkyl chain of 1 to 8 carbon atoms and R¹ is an alkyl chain of 6 to 30 carbon atoms and X is an anion.

The term “confectionery composition” as used herein includes within its meaning chewing gum, and orally soluble tablets, beads and lozenges. The confectionery compositions of the present invention are portable and can be packaged and stored in a consumers pocket or purse for consumption anytime and anywhere. Due to the inherent nature of the saliva dissolvable lozenge or chewable gum product, prolonged contact with the tooth surfaces is achieved when the confectionary composition is used so that the delivery of the antibacterial ester in lozenge tablet, bead or chewing gum form insures that an adequate dosage of the antiplaque ester is deliverable when the product is used.

When the confectionery composition of the present invention is placed within the mouth and chewed or slowly dissolves, an effective antiplaque amount of the antibacterial ester is released from the composition into the saliva where it can reach the surface of the teeth to prevent plaque accumulation. By consistent daily use of the confectionary composition of the present invention the consumer will then obtain maximum plaque reduction from the teeth.

### DESCRIPTION OF PREFERRED EMBODIMENTS

Antibacterial Ester

In the above identified antibacterial ester formula, R²CO may be a natural system mixed fatty acid residue such as coconut oil fatty acid tallow fatty acid residue, and the like, or a mono-fatty acid residue such a lauroyl, myristyl, stearoyl and the like, the lauroyl group being preferred.

Examples of antibacterial ester salts of the above identified formula include an inorganic acid salt such as hydrochloride, sulfate or an organic salt such as acetate, tartarate or citrate, the chloride salt being preferred.

Examples of antibacterial ester compounds preferred in the practice of the present invention are antibacterial ester compound of the above-identified formula wherein n in the formula equals 3 useful in the practice of the present invention include N^{α}-cocoyl-L-arginine propyl ester, N^{α} stearoyl-L-arginine methyl ester, N^{α} steaoryl-L-arginine ethyl ester hydrochloride. The term “cocoyl” is an abbreviation for coconut oil fatty acid residue, and chloride salts of these ester compounds hereinafter being referred to as arginine derivative compounds. The salt of the arginine derivative compound, ethyl lauroyl arginine, is preferred for use in the practice of the present invention.

The antibacterial ester of the present invention is present in the aqueous oral compositions of at a concentration of about 0.05 to about 20% by weight and preferably about 0.75 to about 5% by weight.

Confectionary Vehicle

The antibacterial ester compound of this invention can be incorporated in lozenges, beads, tablets or in chewing gum or other similar solid delivery systems or vehicles by stirring the compound into a warm base with flavor, non-cariogenic sweeteners such as sorbitol and the like.

Lozenge/Bead/Tablet

The vehicle or carrier in a lozenge bead or tablet is a non-cariogenic, solid water-soluble polyhydric alcohol (polyol) such as mannitol, xylitol, sorbitol, malitol, hydrogenated starch hydrozylate, hydrogenated glucose, hydrogenated disaccharides or hydrogenated polysaccharides, in an amount of about 85 to about 95% by weight of the total composition. Emulsifiers such as glycerin, and tableting lubricants, in minor amounts of about 0.1 to 5% by weight, may be incorporated into the tablet, bead or lozenge formulation to facilitate the preparation of the tablet beads and lozenges. Suitable lubricants include vegetable oils such as coconut oil, magnesium stearate, aluminum stearate, talc, starch and Carbowax. Suitable non-cariogenic gums include kappa carrageenan, carboxymethyl cellulose, hydroxyethyl cellulose and the like.

The lozenge, bead or tablet may optionally be coated with a coating material such as waxes, shellac, carboxymethyl cellulose, polyethylene/maleic anhydride copolymer or kappa-carrageenan to further increase the time it takes the tablet or lozenge to dissolve in the mouth. The uncoated tablet or lozenge is slow dissolving, providing a sustained release rate of active ingredients of about 3 to 5 minutes. Accordingly, the solid dose tablet, bead and lozenge compositions of this invention affords a relatively longer time period of contact of the teeth in the oral cavity with the antibacterial ester compound.

Sweeteners

The sweetening agent ingredient used in the practice of the present invention include sweeteners such as artificial sweeteners including as sodium or calcium saccharin salts, cyclamate salts, such as the sodium salt and the like, and the free acid form of saccharin; dipeptide based sweetening agents such as L-aspartyl-L-phenyl-alanine methyl ester, dihydrochalcone; glycyrrhizin; and the synthetic sweetener 3,6-dihydro-6-methyl-1,1,2,3-oxathiazin-4-one-2,2-dioxide, particularly the potassium (Acesulfame-K), sodium and calcium salts. The polyols of 5 to 12 carbon atoms substituted with 5 to 9 hydroxyl groups such as sugar alcohols including xylitol, sorbitol, and maltitol. Sugar alcohols provide bulk or texture to the compositions of the present invention and are utilized in amounts of about 25% to about 90% by weight preferably about 40% to about 85% by weight. Artificial sweeteners are present in the confectionery compositions of the present invention at a concentration of about 0.1 to about 1% by weight.

In a preferred embodiment of this invention, the sweetening agent used is a combination of an artificial sweetener such as aspartame, the artificial sweetener being present generally in amounts of about 0.05% to about 2.0% by weight and preferably about 0.1% to about 1.0% by weight and the sugar alcohol or polyol is present in the lozenge, bead or tablet at a concentration of about 40% to about 60% by weight, preferably about 45% to about 55% by weight of the polyol sweetener.

Flavoring Agents

One or more flavoring agents are used in the confectionary composition of this invention. A variety of flavors known in the art may be used, including essential oils, such as cinnamon, spearmint, peppermint, menthol, birch, anise wintergreen oil and eucalyptus oil. Natural fruit flavors derived from the essence of fruits, such as apple, pear, peach, strawberry, cherry, apricot, orange, watermelon, banana and the like; bean derived flavors such as coffee, cocoa and the like; wine derived curacao zin and the like, and pungent materials, such as affinin, pepper, mustard and the like. Flavoring agents are incorporated in the confectionery compositions of the present invention at a concentration of about 0.5 to about 5% by weight and preferably about 1.0 to about 3.0% by weight.

Other Ingredients

Tableting lubricants, in minor amounts of about 0.1 to about 2.0% by weight may be incorporated in the tablet, bead or lozenge formulation to facilitate the preparation of both the tablets, beads and lozenges. Suitable lubricants include vegetable oils, such as coconut oil, magnesium stearate, aluminum stearate, talc, starch and Carbowax.

Chewing Gum

The chewing gum of the present invention is preferably a sugarless chewing gum containing the antibacterial ester compound. Chewing gum formulations in which the antibacterial ester of the present invention may be incorporated are well known in the art and typically contain, in addition to, a chewing gum base, one or more plasticizing agents; at least one sweetening agent and at least one flavoring agent.

Gum base materials suitable for use in the practice of this invention are well known in the art and include natural or synthetic gum bases or mixtures thereof. Representative natural gums or elastomers include chicle, natural rubber, jelutong, balata, guttapercha, lechi caspi, sorva, guttakay, crown gum, perillo, or mixtures thereof. Representative synthetic gums or elastomers include butadiene-styrene copolymers, polyisobutylene and isobutylene-isoprene copolymers.

The gum base is incorporated in the chewing gum product at a concentration of about 10 to about 40% by weight and preferably about 20 to about 35% by weight.

Plasticizing/softening agents commonly used in chewing gum compositions are suitable for use in this invention, including gelatin, waxes and mixtures thereof in amounts of 0.1 to 5% by weight.

The sweetening agent ingredient used in the practice of this invention may be selected from a wide range of materials, and include the same artificial and polyol sweeteners used for the preparation of tablets, beads and lozenges. Polyol sweeteners such as sorbitol and malitol are present in the chewing gum composition of the present invention in amounts of about 40 to about 80% by weight and preferably about 50 to about 75% by weight. The artificial sweetener is present in the chewing gum composition of the present invention in amounts of about 0.1 to about 2% by weight and preferably about 0.3 to 1% by weight.

In addition to the ingredients listed above, the gum compositions may also include additives such as colorants, flavoring agents and the like. For example, titanium dioxide may be utilized as a colorant. A variety of flavors known in the art may be used, including essential oils, such as cinnamon, spearmint, peppermint, menthol, birch, anise and the like; natural fruit flavors derived from the essence of fruits, such as apple, pear, peach, strawberry, cherry, apricot, orange, watermelon, banana and the like; bean-derived flavors, such as coffee, cocoa and the like. Flavoring agents are incorporated in the chewing gum formulation at a concentration of about 0.5 to about 5% by weight and preferably 1 to 3% by weight.

Antitartar agents compatible with antibacterial esters such as ethyl lauroyl arginine may also be included in the oral composition of the present invention. An example of such antitartar agents include cationic polyphonates such as water soluble quaternary aminoalkylene phosphonic compounds as disclosed in U.S. Pat. No. 4,118,472, the disclosure of which is herein incorporated by reference. These antitartar agents may be included in the oral composition of the present invention at a concentration of about 0.1 to about 5% by weight.

Antitartar agents which are not compatible with antibacterial esters such as pyrophosphate and polyphosphate salts may be included in one component of a dual component oral composition system in which a first component contains the antibacterial ester and the second component contains the incompatible antitartar salt, the first and second components being maintained separate from each other until dispersed and combined for application to the teeth.

Manufacture

The confectionary composition of the present invention is made by any suitable conventional process where the antibacterial ester compound is incorporated into the solid base material such that no water or a limited amount of ingredients that absorb water are used that would result in undesirable amounts of water being introduced into the composition during processing or storage.

Equipment and processing techniques have been well developed in the art for preparing packaging chewing gum, tablets, beads and lozenges.

One method for manufacturing the composition of the invention comprises first heating the base material to a temperature sufficient to drive off any water in the composition. The base material is then cooled to a temperature at which the antibacterial ester and other temperature sensitive ingredients such as plasticizers and sweeteners are incorporated and mixed into the base gum or sweetener vehicle.

The tablets of the present invention are conventionally made by grinding the ingredients once mixed and then compressing or molding the ingredients to form a suitable means for the delivery of the antibacterial ester compound. In order to produce tablets it is necessary to have a free flowing material which has good self binding properties and which will not stick to the molding or compression equipment.

An illustrative procedure for formulating the chewing gum composition is as follows: the gum base is first melted in a heated kettle at 55-65° C. One or more of the sweeteners are then added to the gum base followed by one or more flavors, plasticizer. All ingredients are then mixed for a sufficient period of time to ensure adequate dispersion. The mixture is then allowed to cool and the antibacterial ester compound is added thereafter the solid, cooled material is cut into suitable serving sizes.

In order to enhance shelf stability, in addition to the admixture used in the preparation of the chewable product being substantially free of water, the finished product should be packaged in a manner so as to minimize exposure to air and moisture.

The following Examples are illustrative of the present invention, but it is understood that the invention is not limited thereto.

### EXAMPLE I

LOZENGE

| Ingredient | Wt. % |
|---|---|
| Saccharin | 0.15 |
| Magnesium Stearate | 0.40 |
| Glycerin | 1.0 |
| Ethyl lauroyl arginine | 0.5 |
| Flavor | 2.0 |
| Sorbitol | Q.S. |

### EXAMPLE II

BEAD

| Ingredient | Wt. % |
|---|---|
| Gelatin | 30 |
| Flavor | 45 |
| Vegetable oil | 22.5 |
| Aspartame | 0.2 |
| Ethyl lauroyl arginine | 1 |
| Food color | 0.002 |
| Flavor | 2.0 |
| Ethyl alcohol | 0.3 |
| Water | Q.S. |

### EXAMPLE III

TABLET

| Ingredient | Wt. % |
|---|---|
| Starch coated dicalcium phosphate | 40 |
| Cellulose | 20 |
| Glycerin | 12 |
| Sorbitol | 17 |
| Sodium saccharin | 0.2 |
| Flavor | 1 |
| Lecithin | 0.5 |
| Ethyl lauroyl arginine | 0.5 |
| Water | Q.S. |

### EXAMPLE IV

CHEWING GUM

| Ingredient | Wt. % |
|---|---|
| Gum base | 25 |
| Binder | 10 |
| Aspartame | 0.5 |
| Ethyl lauroyl arginine | 1 |
| Flavor | 2.0 |
| Titanium dioxide | 0.4 |
| Sorbitol/maltitol (50:50) | Q.S. |

What is claimed is:

1. A confectionery composition delivering antiplaque agents to the oral cavity when applied thereto, the composition being comprised of a homogeneous mixture of a solid base material, a sweetener and an antibacterial ester having the formula 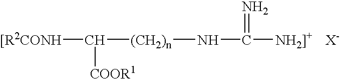
wherein R is an alkyl chain of 1 to 8 carbon atoms and R¹ is an alkyl chain of 6 to 30 carbon atoms and X is an anion.

2. The confectionery composition of claim 1 wherein the antibacterial agent is the chloride salt of ethyl lauroyl arginine.

3. The confectionery composition of claim 1 wherein the antibacterial agent is present at a concentration of about 0.05 to about 20% by weight, and preferably about 0.75% to about 5%.

4. The confectionary composition of claim 1 wherein the composition is a chewing gum.

5. The confectionary composition of claim 1 wherein the composition is a tablet.

6. The confectionary composition of claim 1 wherein the composition is a lozenge.

7. The composition of claim 1 wherein the composition is a bead.

8. The composition of claim 1 wherein the composition is an oral spray
